(19) United States
(12) Patent Application Publication
(10) Pub. No.: US 2026/0224917 A1
(43) Pub. Date: Aug. 6, 2026

(54) SYSTEMS AND METHOD OF HISTOTRIPSY DIABETES THERAPY

(71) Applicant: HISTOSONICS, INC., Ann Arbor, MI (US)

(72) Inventor: Joshua Stopek, Minneapolis, MN (US)

(21) Appl. No.: 19/466,145

(22) Filed: Jan. 30, 2026

Related U.S. Application Data
(60) Provisional application No. 63/752,425, filed on Jan. 31, 2025.

(51) Int. Cl. A61N 7/00

(52) U.S. Cl. CPC A61N 7/00; A61N 2007/0004; A61N 2007/0039; A61N 2007/0052; A61N 2007/0086

(57) ABSTRACT

A system and method for applying histotripsy therapy, including an acoustic coupling assembly for placement on the patient, a therapy transducer configured to emit an ultrasound signal and generate a bubble cloud at a focal point, wherein the therapy transducer is configured to acoustically couple to tissues of the duodenum of the patient via the acoustic coupling assembly and fluid within the duodenum of the patient, and an application configured to receive images of the duodenum of the patient, define an inner layer of the duodenum, define a therapy cylinder, define a plurality of bubble cloud locations matching a periphery of the therapy cylinder, and robotically drive the therapy transducer to a position and orientation where a focal point of the therapy transducer matches the bubble cloud locations, and apply histotripsy therapy from the therapy transducer to form a bubble cloud at each of the defined bubble cloud locations.

### FIELD

This disclosure is directed to systems and methods of planning and conducting diabetes therapy using local high intensity therapeutic ultrasound (HITU).

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, histotripsy realm; it is necessary to deliver acoustic energy in the form of high amplitude acoustic pulses with low duty cycle.

Compared with conventional focused ultrasound technologies, histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) histotripsy produces lesions in a controlled and precise manner. It is important to emphasize that unlike thermal ablative technologies such as microwave, radiofrequency, high-intensity focused ultrasound (HIFU), cryogenic, or radiation, histotripsy relies on the mechanical action of cavitation for tissue destruction and not on heat, cold or ionizing energy.

### BRIEF DESCRIPTION OF THE DRAWINGS

The features of the disclosure are set forth with particularity in the claims that follow. A better understanding of the features and advantages of the disclosure will be obtained by reference to the following detailed description that sets forth illustrative embodiments, in which the principles of the disclosure are utilized, and the accompanying drawings of which:

FIG. 1A is a perspective view of a histotripsy system in accordance with the disclosure;

FIG. 1B depicts a therapy transducer and imaging transducer in accordance with the disclosure;

FIG. 2 depicts a histotripsy system deployed to treat a patient in accordance with the disclosure;

FIG. 3 is a plot of a pressure wave during application of a histotripsy ultrasound pulse in accordance with the disclosure;

FIG. 4 is a flow diagram for a method of application of histotripsy therapy to the duodenum of a patient;

FIG. 5 is a schematic depicting a ballon catheter placed within the duodenum of the patient for application of histotripsy therapy;

FIG. 6 is a cross-sectional view of a therapy catheter inserted within the duodenum of the patient for application of histotripsy therapy; and

FIG. 7 is a cross-sectional view of a second therapy catheter inserted within the duodenum of the patient for application of histotripsy therapy.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing, and ablation. Furthermore, due to tissue selective properties, histotripsy may be used to create a cytoskeleton that allows for subsequent tissue regeneration either de novo or through the application of stem cells and other adjuvants. Histotripsy can also be used to cause the release of delivered agents such as chemotherapy and immunotherapy by locally causing the release of these agents by the application of acoustic energy to the targets. As will be described below, the acoustic cavitation system may include various sub-systems, including a cart, therapy, integrated imaging, robotics, coupling and software subsystems. The acoustic cavitation system also may comprise various other components, ancillaries, and accessories, including but not limited to computers, processors, memory, software, applications, cables, connectors, networking devices, power supplies, displays, drawers/storage, doors, wheels, and various simulation and training tools, etc. All systems, methods and means of creating/controlling/delivering histotripsy are considered to be a part of this disclosure.

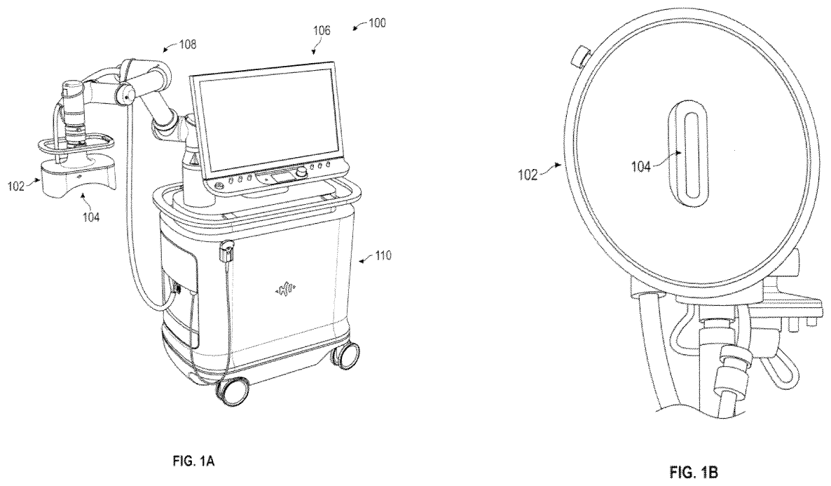

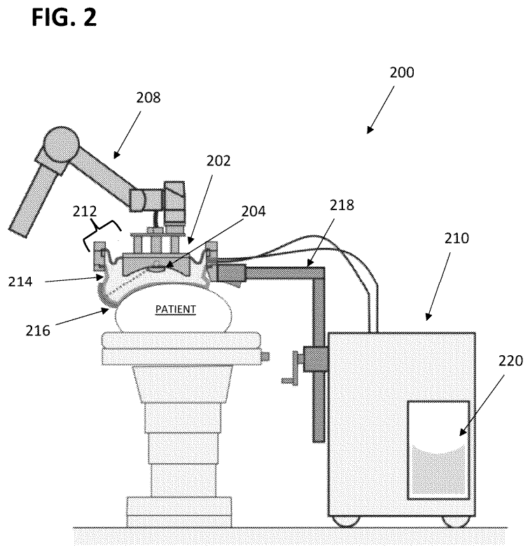

FIG. 1A depicts a histotripsy system 100 in accordance with the disclosure. The histotripsy system 100 includes a therapy transducer 102, an imaging system 104, a display and control panel 106, a robotic positioning arm 108, and a cart 110. The system can further include an ultrasound coupling interface and a source of coupling medium (FIG. 2).

FIG. 1B is a bottom view of the therapy transducer 102 and the imaging system 104. As shown, the imaging system 104 can be positioned in the center of the therapy transducer 102. However, other embodiments can include the imaging system 104 positioned in other locations within the therapy transducer 102, or even directly integrated into the therapy transducer 102. In some embodiments, the imaging system is configured to produce real-time imaging at a focal point of the therapy transducer 102. The system also allows for multiple imaging transducers 104 to be located within the therapy transducer 102 to provide multiple views of the target tissue simultaneously and to integrate these images into a single 3-D image. In some embodiments, the imaging system and capability may comprise high-frequency or ultrahigh-frequency ultrasound imaging which may further enable imaging various tissue layers including those layers/interfaces desired for treatment delivery. The system and system software may display live streaming images of such layers and in context to other therapy planning features (target crosshairs, treatment plan contours, safety planes, etc.).

The histotripsy system 100 may comprise, and the cart 110 may enclose, one or more of various sub-systems, including a therapy sub-system that can create, apply, focus and deliver acoustic cavitation/histotripsy through one or more therapy transducers 102, an integrated imaging sub-system (or connectivity thereto) allowing real-time visualization and display of the treatment site and histotripsy effect through-out the procedure (e.g., via, a robotics positioning sub-system to mechanically and/or electronically steer the therapy transducer) and further enabled to connect/support or interact with a coupling sub-system to allow acoustic coupling between the therapy transducer 102 and the patient, and software to communicate, control and interface with the system and computer-based control systems (and other external systems) and various other components, ancillaries and accessories, including one or more user interfaces and displays, and related guided work-flows, all working in part or together. The histotripsy system 100 may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described in greater detail below, the cart 110 may be configured and arranged to be used in a radiology environment and in some cases in concert with imaging (e.g., fluoroscopy, augmented fluoroscopy, computed tomography (CT), cone beam CT and/or magnetic resonance imaging (MRI) scanning). In other embodiments, the cart 110 may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The cart 110 may also work with a patient surface (e.g., table or bed) to allow the patient to be presented and repositioned in a variety of positions, angles and orientations, including allowing changes to such to be made pre, peri and post-procedurally. The cart 110, and subsystems thereof, may further comprise the ability to interface and communicate with one or more external imaging or image data management and communication systems, not limited to ultrasound, CT, fluoroscopy, cone beam CT, positron emission tomography (PET), PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri, intra and/or post histotripsy) and to provide access to and display of patient medical data including but not limited to laboratory and historical medical record data.

In some embodiments one or more carts may be configured to work together. As an example, one cart may comprise a bedside mobile cart equipped with one or more robotic arms enabled with a therapy transducer, and therapy generator/amplifier, etc., while a companion cart working in concert and at a distance of the patient may comprise integrated imaging and a console/display for controlling the robotic and therapy facets, analogous to a surgical robot and master/slave configurations.

FIG. 2 illustrates one embodiment of a histotripsy therapy and imaging system 200, including a coupling assembly 212. As described above, a histotripsy therapy and imaging system can include a therapy transducer 202, an imaging system 204, a robotic positioning arm 208, and a cart 210.

The therapy and/or imaging transducers are placed in the coupling assembly 212 which can further include a coupling membrane 214 and a membrane constraint 216 configured to prevent the membrane from expanding too far from the transducer. The coupling membrane 214 is filled with an acoustic coupling medium such as a fluid or a gel. The membrane constraint 216 can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 212 is supported by a mechanical support arm 218 which can be load bearing in the x-y plane but allow for manual or automated z-axis adjustment. The mechanical support arm 218 can be attached to the floor, the patient table, or the cart 210. The coupling assembly 212 is designed and configured to conform and hold the coupling membrane 214 in place against the patient's skin while still allowing movement of the therapy/imaging transducer 202, 204 relative to the patient the coupling membrane 214 with the robotic positioning arm 208.

The system can further include a fluidics system 220 that can include a fluid source, a cooling and degassing system, and a programmable control system. The fluidics system is configured for external loading of the coupling assembly 212 with automated control of fluidic sequences so that the coupling membrane 214 can conform around the patient.

The system can further include an endoscope and/or endoscopic navigation system including optical imaging and means of tracking, including but not limited to relative or absolute tracking in multiple degrees of freedom (DoF), further comprising either optical or electromagnetic shape sensing. This may include 6 DoF absolute shape sensing and in some embodiments may comprise a full 6DoF shape sensing endoscope with integrated high-frequency ultrasound imaging and histotripsy, which may be user-guided (manual) and/or robotically-assisted.

### Histotripsy

Histotripsy is achieved by generating short, high amplitude, focused ultrasound pulses to generate a dense, energetic, “bubble cloud,” capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically affect tissue structure, and in some cases liquefy, suspend, solubilize and/or destruct tissue into sub-cellular components.

Histotripsy can be applied in various forms, including: 1) intrinsic-threshold histotripsy which delivers pulses typically with a 1-2 cycles of high amplitude negative/tensile phase pressure exceeding the intrinsic threshold to generate cavitation in the medium (e.g., ~24-28 MPa for water-based soft tissue), 2) shock-scattering histotripsy which delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) boiling histotripsy which employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous, and robotically mediated surgical procedures. It may be further targeted, planned, directed, and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism.”

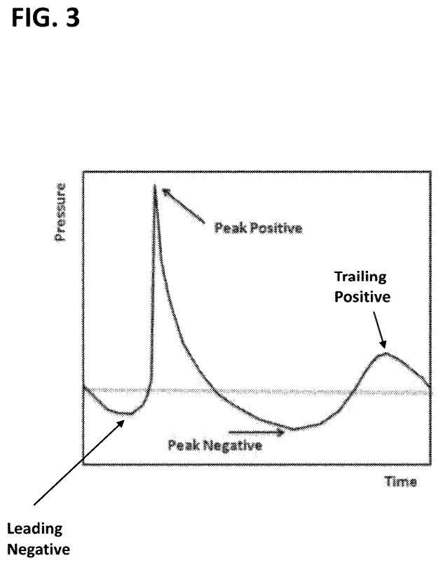

FIG. 3 illustrates an ultrasound pulse that can be used for shock scattering histotripsy. As shown the ultrasound pulse can include a leading negative half cycle, a peak positive half cycle, a peak negative half cycle, and a trailing peak positive half cycle (with the pulse traveling from right to left on the page). As shown, the trailing peak positive cycle has a lower amplitude than the peak positive cycle. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface if the amplitude of those cycles is sufficient, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When the amplitude(s) of positive half cycle(s) of each pulse are limited, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism.”

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency,” “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Diabetes Therapy

Diabetes and particularly Type-2 diabetes is one of the most common maladies affecting adults worldwide as they age. Diabetes, and particularly Type-2 diabetes can be caused by insulin resistance. The insulin resistance causes insulin-sensitive tissues in the body to fail to respond normally to insulin. Risk factors for insulin resistance, and in some instances the causes of Type-2 diabetes, include obesity, lack of physical activity, diet, hormonal imbalances, genetics, heart conditions, and even some medications. The acute effects of insulin resistance and Type-2 diabetes can include entering a hyperosmolar hyperglycemic state (HHS), whereby blood sugar levels are very high (over 600 milligrams per deciliter or mg/dL) for a long period, leading to severe dehydration and confusion. On the opposite end of the spectrum of acute effects, where patients are prescribed insulin, severe low-blood sugar (hypoglycemia) can result in blurred or double vision, clumsiness, disorientation, and seizures. While there are many causes of insulin resistance, the underlying process is still not completely understood.

What is better understood is the actions of the stomach and intestines in controlling nutrient absorption, including signaling functions related to blood glucose control. The small intestine, specifically enterocytes and enteroendocrine cells (EEC's) within the duodenum, regulates nutrient absorption, senses the presence and amount of, for example carbohydrates, and signals (e.g., neural and hormonal signaling) that trigger downstream organ (e.g., pancreas, liver, stomach and brain) responses for absorption and storage of glucose throughout the body. It is believed that poor signaling from EEC's, which are found in the mucosa of the duodenum, which may in part be the result of inflammation of duodenal tissue results in improper the improper glucose regulation (e.g., insulin resistance) and thus contributes to the Type-2 diabetes.

Traditional therapies for Type-2 diabetes are primarily focused on diet and exercise regimen along with medications such as Metformin and of course synthetic insulin to help regulate blood glucose levels. These therapies, however, have minimal if any impact on EEC's of the mucosa. A more recently developed technique called duodenal mucosal resurfacing (DMR) seeks to ablate duodenal mucosal tissue and promote regeneration of the mucosa (also called re-epithelialization). The aim of DMR is to stimulate the removal of morphologically and or functionally impaired duodenal mucosa and the subsequent regeneration of normal duodenal epithelium. Effectively killing ineffective EEC's and portions of the mucosa, allowing the body to eliminate those killed cells, and stimulate the regeneration of the epithelium and new EEC's. As will be appreciated this is done while limiting damage to the submucosa and preventing damage to the muscularis layers of the duodenum. Existing techniques for DMR employ either hydrothermal ablation, radio frequency (RF) ablation. Due to the heating generation of these two techniques, there are multiple drawbacks. With respect to RF, lower power RF must be employed to limit the depth of therapy, and further suction is required to ensure good contact with the RF electrodes. Despite these steps an incomplete ablation may still result due to the low power and the undulating surface of the epithelium. With regards to hydrothermal ablation, saline must be injected into the submucosa to create a thermal barrier. In the event this fails, the submucosa of the duodenum can be damaged with negative health effects for the patient. Further both suction and saline injection additional tools to be navigated within the duodenum and appropriately placed which complicates the procedure. Aspects of this disclosure seek to address these shortcomings of existing therapy systems.

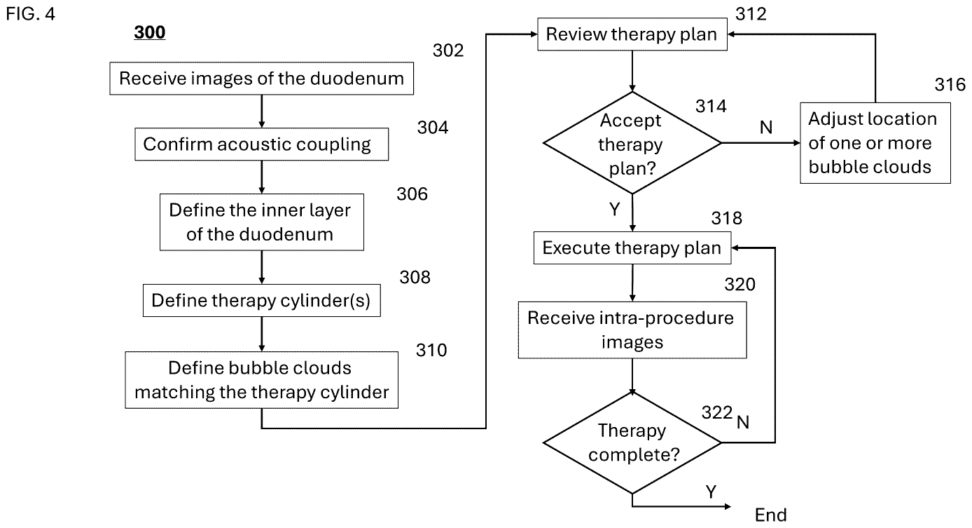

This disclosure contemplates a variety of systems and methodologies for application of histotripsy therapy to the duodenum for the performance of a DMR procedure. Referring to FIG. 4, a first method 300 starts with receiving images via the imaging system 204 of the duodenum at step 302. The acquired images are loaded into a planning application stored in a memory and executed by a processor in a computing device. The computing device may be on the cart 110 and associated with the control panel 106 or may be a separate computer such as a laptop or a tablet configured to receive the images. At step 304, analysis of the images (either automatically by the application or manually by the clinician) confirms that the patient has ingested sufficient quantity of a coupling medium (e.g., degassed water, saline, etc.) based on the acoustic coupling if the imaging system 204 with the tissues of the duodenum visualizing the duodenum with the imaging system 204. An inner layer of the duodenum (e.g., the epithelium or the entire mucosa) may be identified in the acquired images at step 306. Defining the inner layer may be automatically performed via image processing methods such as segmentation, edge detection, thresholding, and others, or may be manually performed by the clinician using tools within the application. With the duodenum being substantially cylindrical, a therapy cylinder or a series of interconnected therapy cylinders, larger (e.g., 1-2 mm) than the inner diameter of the duodenum is defined at step 308. A plurality of overlapping bubble clouds is defined at step 310 that substantially match the periphery of the therapy cylinder(s). Each bubble cloud defined in step 310 is associated with a position, orientation, and of the robotic positioning arm 208 and the therapy transducer 202. The combination of the therapy cylinders, the overlapping bubble clouds, and the positions and orientations of the therapy transducer 202 and robotic positioning arm 208 define a therapy plan and at step 312 the therapy plan can be reviewed to ensure that none of the bubble clouds extend into the submucosa or other tissues to be spared during the procedure. At step 314 a determination is made whether the therapy plan is accepted, if not the method moves to step 316 where the location of one or more bubble clouds is adjusted and the method returns to step 312. If the therapy plan is accepted, the method moves to step 318 where the therapy plan is executed. Execution of the therapy plan results causes an application on the computing device to drive the robotic positioning arm 208 and therapy transducer 202 to a position and orientation such that the focal point of energy emitted from the therapy transducer 202 is at a center of each bubble cloud for a duration sufficient to solubilize and lyse the tissues defined in the therapy cylinder. Histotripsy therapy is thus applied to the therapy cylinder(s) along the length of, or a defined portion of, the duodenum.

During the execution of the therapy plan intra-procedural images are acquired (e.g., with the imaging system 204) at step 320. At any time during the application of histotripsy therapy to the therapy cylinder if an issues is observed, a stop button associated with the control panel 106 can be depressed to halt the procedure. In the ordinary course, as the therapy plan is executed and the intraprocedural images are acquired, and periodically a determination is made whether the therapy is complete 322. If the therapy is not complete the method returns to step 318 for execution of the therapy plan, but if the therapy is complete the method ends.

As will be appreciated, by application of histotripsy therapy to the therapy cylinder the mucosa, (e.g., the epithelium of the duodenum) is liquified and lysed painlessly and without the generation of heat in the duodenum. Moreover, by adjusting the magnitude, frequency, and duty cycle of the histotripsy signal, a high degree of tissue selectivity is possible. The tissue selectivity enables the tuning of the therapy transducer 202 such that cells of the submucosa and further mitigates and potentially eliminates the damaging of the submucosa and muscularis layers of the duodenum. In some embodiments, the histotripsy pulse sequence, waveform, and treatment protocol may be configured to specifically exert desired tissue effects for these applications, and in some cases, executed as a preconfigured treatment protocol (e.g., not requiring the user to adjust specific therapy parameters).

Following therapy and after a period of time for the treated tissues of the patient to initiate a healing response, the mucosa (e.g., of the epithelial cells and particularly the EEC's), the patient's duodenum begin to regrow. Examination via an endoscopic procedure will reveal that the mucosa has shed all of the cells killed by the histotripsy procedure and new healthy cells have regrown. In some instances, portions of the duodenum may appear not to have received therapy. If desired, or if the area is large enough the procedure may be repeated focusing on those area which have not received prior therapy.

As described herein, method 300 relies on bodily function and the ingestion of acoustic coupling media to ensure good acoustic coupling between the therapy transducer 202 and the tissues liming the duodenum. This disclosure is not so limited, and rather than rely on the bodily functions to ensure sufficient coupling media is retained within the duodenum to apply non-invasive histotripsy (e.g., via a histotripsy therapy and imaging system 200) additional systems are contemplated within the disclosure to ensure sufficient coupling medium for the application of histotripsy therapy.

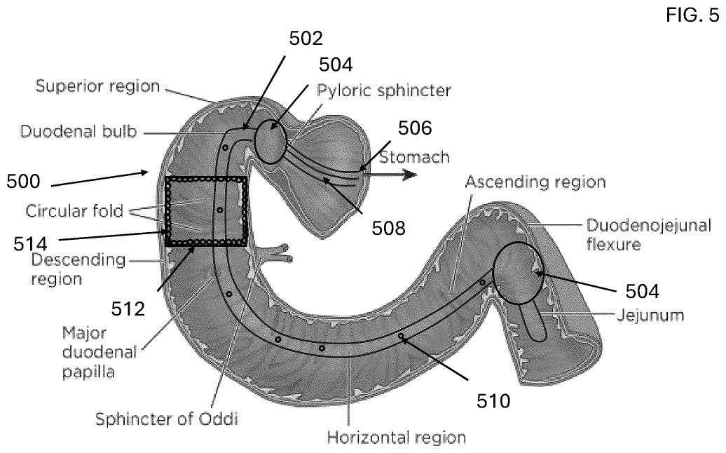

FIG. 5 depicts a balloon catheter 502 having been placed within the duodenum 500 of a patient. In utilizing the balloon catheter 502, the balloon catheter 502 is inserted through the mouth of the patient, down the esophagus, through the stomach and through the duodenum such that a distal end of the catheter 502 is located near the Jejunum. A first balloon 504 is depicted inflated at or near the pyloric sphincter and a second balloon 504 is inflated at the distal end of the duodenum (e.g., at the Jejunum). In some instances, the proximal balloon 504 is omitted from the catheter 502, relying on normal functions of the pyloric sphincter and the distal balloon 504 to retain sufficient coupling medium within the duodenum. Placement of the balloons 504 can be confirmed via imaging such as ultrasound, high-frequency/ultrahigh-frequency ultrasound, fluoroscopy, or even cone-beam CT imaging. Whether one or two balloons are employed, openings 510 formed in the catheter 502 and fluidly connected to a channel 506 of the catheter 502, which itself is connected to a source of coupling medium (e.g., fluidics system 220) enabling the injection of coupling medium into the duodenum. The coupling media applies pressure to the tissues of the walls of the duodenum. A sensor, not shown, connected to the catheter 502 and the fluidics system 220 monitors the pressure of fluid injected into the duodenum to ensure that it is not over pressurized. The balloons 504 may be connected to a second channel 506 which itself may be connected to an inflation medium. The inflation medium may be air, or a liquid such as the coupling medium from the fluidics system.

With the catheter 502 placed within the duodenum of the patient and the balloons 504 placed at desired locations as confirmed via imaging, the balloons 504 are inflated. After inflation of the balloons, method 300 may be undertaken, and following termination of method 300 the catheter 502 can be deflated and removed from the patient.

Regardless of whether the catheter 502 is employed in the application of histotripsy therapy to the duodenum, FIG. 5 depicts a therapy cylinder 512 which substantially conforms to the inner diameter of the walls of the duodenum for a given length of the duodenum. A series of bubble cloud locations 514 along the periphery of the therapy cylinder 512 are the locations at which the focal point of the therapy transducer 202 is to be positioned so that histotripsy therapy can be applied such that just the mucosa is treated and the submucosa and the muscularis are unaffected by the therapy. The duodenum 500 of FIG. 5 is depicted in cross section and only the outline of the therapy cylinder 512 is shown with bubble cloud locations 514, however one of skill in the art will recognize that the entire surface of the mucosa that is defined within the therapy cylinder 512 would have bubble cloud locations 514. Here the bubble cloud locations 514 within the therapy cylinder 512 are omitted for clarity purposes. The overlap of bubble cloud locations and tissue layers may be configured by the machine and/or user in some embodiments, with pre-defined rules/logic around how much a cloud location may intersect with various defined tissue layers.

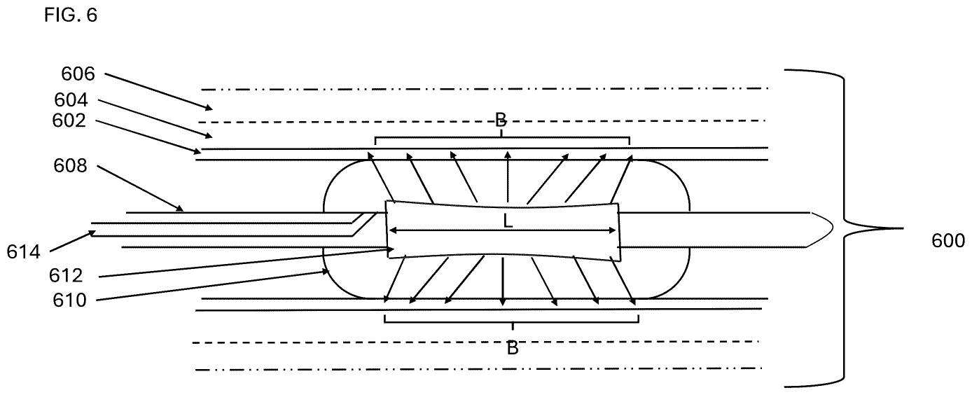

Heretofore the systems and methods described are related to the use of the non-invasive histotripsy therapy system 200. However, the disclosure is not so limited. FIG. 6 depicts a cross-sectional view of the duodenum 600. The duodenum, as described above, includes multiple layers including the mucosa (epithelial layer) 602, the submucosa 604, and the muscularis layers 606. In accordance with the disclosure a histotripsy catheter 608 including a balloon 610 and a therapy transducer 612 secured thereto. The balloon 610 receives coupling medium from the fluidics system 220 via channel 614. The balloon 610 ensures that the therapy transducer 612 is centered within the duodenum 600, further the coupling medium within the balloon ensures acoustic coupling between the therapy transducer 612 and the tissues of the duodenum 600, particularly the mucosa 602. In some embodiments, the therapy transducer may comprise an integrated ultra-high frequency imaging probe. In some embodiments, the imaging transmits/receive signals may be synchronized with the therapy signals.

As will be appreciated, the therapy transducer 612 can be electronically aimed or steered such that acoustic energy (shown graphically as arrows) can be directed to impact the mucosa 602 of the duodenum 600. The range of this electronic or steering defines the length of the mucosa 602 of the duodenum 600 that can receive therapy with the catheter 608 held stationary by the balloon 610. In the embodiment depicted in FIG. 6, the therapy transducer 612 is a fixed omnidirectional transducer capable of generating a focal point at wide angles from the longitudinal axis of the catheter 6108 along the length L of the therapy transducer 612. Thus, in practice by electronically steering or focusing the therapy transducer, a focal point and bubble cloud can be formed circumferentially on the mucosa 602 of the duodenum and along with a defined length of the duodenum 600. In this manner, a band B of applied therapy is defined both circumferentially within the duodenum 600 and along a length of the duodenum 600. By applying therapy to multiple of such bands B therapy, the entirety of the duodenum, or a desired portion thereof can receive histotripsy therapy.

In practice, and under visualization (e.g., ultrasound, fluoroscopy, ultrasound, CBCT, etc.) the catheter 608 is navigated step wise through the duodenum 600. At each stopping point, histotripsy therapy is applied and a band B of therapy is defined. The balloon 610 is then slightly deflated, the catheter 608 advanced to the subsequent location along the length of the duodenum 600, histotripsy therapy applied, and as second band B of therapy is defined. This process continues until the entirety of the desired portion of the duodenum 600 receives histotripsy therapy. Once therapy is complete the balloon 610 is deflated and the catheter 608 is removed from the patient.

Alternatively, the therapy transducer 612 may not be omni direction, but rather may define a single direction for application of acoustic energy. The therapy transducer 612 is then rotatable about the longitudinal axis of the catheter 608. Thus, the therapy transducer 612 is both electronically focused and steered and also mechanically rotated to define a band B of therapy.

Regardless of which therapy transducer 612 is employed, based on the volume of fluid used to fill the balloon 610, and the properties of the balloon 610, a determination is made as to the inner diameter of the duodenum 600 at the location of inflation. With the diameter determined, a focal length of the therapy transducer may be adjusted such that when activated a bubble cloud forms at the epithelial layer (e.g., only in the mucosa 602) of the duodenum 600.

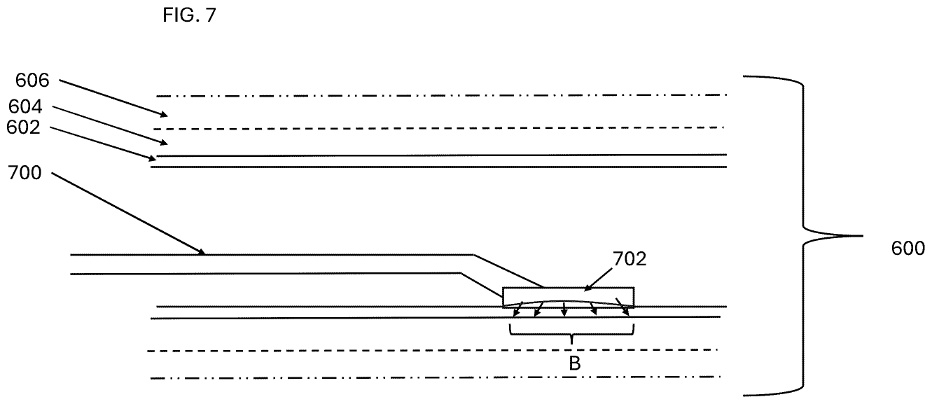

Yet a further catheter 700 for direct application of histotripsy therapy to the mucosa 602 of the duodenum 600 is depicted in FIG. 7. The catheter 700 includes a therapy transducer 702 that can be directly applied to the mucosal layer 602 of the duodenum 60. The focal length of the direct application therapy transducer 702 is very short (e.g., 1-2 mm). Thus, application of histotripsy is limited to just mucosa and preferably the epithelial cells of the duodenum 600. The direct application therapy transducer 702 can be rotated within and held against the mucosa 602 therapy is applied. Rotation of the catheter 700 along the inner diameter of the duodenum forms a band B of therapy. After one band B of therapy is formed, the catheter 700 is advanced (or retracted) and a subsequent band B of therapy is applied to the mucosa 602 of the duodenum 600 until a desired length of the duodenum 600 is treated.

Those of skill in the art will recognize that the catheter 700 may include a biasing means (e.g., formed of an elastomeric material) to bias the therapy transducer 702 against the mucosa 602 of the duodenum 600. Further, in some instances, one or more pull-wires (not shown) may be employed to mechanically bias the therapy transducer 702 against the mucosa 602 of the duodenum 600. Still further, the therapy transducer 702 and catheter 700 may include a lumen connected to a vacuum source. Application of vacuum to the lumen causes the therapy transducer 702 to be suctioned to the mucosa 602 of the duodenum 600 to secure and acoustically couple the therapy transducer 702 to the mucosa 602 of the duodenum 600.

Through the methods and systems described herein histotripsy therapy is applied to the small intestine, specifically enterocytes and enteroendocrine cells (EEC's) within the duodenum. The histotripsy therapy kills the cells which regulate nutrient absorption, senses the presence and amount of, for example carbohydrates, and signals (e.g., neural and hormonal signaling) that trigger downstream organ (e.g., pancreas, liver, stomach and brain) responses for absorption and storage of glucose throughout the body. As a result of this targeted killing of these cells, the patient's natural healing response, regenerates the cells of the mucosa and particularly the enterocytes and EEC's. By their regeneration, the body's natural responses to stimuli resulting from ingesting is returned to normal, causing the body's regulatory systems to return to proper glucose regulation (e.g., insulin resistance) and thus reducing and elimination of the causes of Type-2 diabetes.

Described herein are applications operable on one or more computing devices. Those of skill in the art will recognize that the three applications may be modules of a single histotripsy application and that the applications share features which allow aspects of one module to connect to another module without departing from the scope of the disclosure.

### EXAMPLES

### Example 1

The embodiments described above are cited by way of example, and that the present invention is not limited to what has been particularly shown and described hereinabove. Rather, the scope of this disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are considered an integral part of the application.

We claim:

1. A system for applying histotripsy therapy, comprising:
an acoustic coupling assembly configured for placement on a patient;
a therapy transducer configured to emit an ultrasound signal and generate a bubble cloud at a focal point of the therapy transducer, wherein the therapy transducer is configured to acoustically couple to a duodenum of the patient via the acoustic coupling assembly and fluid within the duodenum of the patient; and
an application stored in a memory and when executed by a processor of a computing device cause the computing device to:
receive images of the duodenum of the patient;
define an inner layer of the duodenum;
define a therapy cylinder;
define a plurality of bubble cloud locations matching a periphery of the therapy cylinder; and
robotically drive the therapy transducer to a position and orientation where a focal point of the therapy transducer matches the bubble cloud locations; and
apply histotripsy therapy from the therapy transducer to form a bubble cloud at each of the defined bubble cloud locations.

2. The system of claim 1, wherein the received images are ultrasound images, fluoroscopic images, or cone-beam computed tomography images.

3. The system of claim 1, wherein the therapy cylinder, bubble cloud locations, and positions and orientations of a therapy transducer define a therapy plan.

4. The system of claim 1, further comprising a catheter configured for insertion into the duodenum of the patient.

5. The system of claim 4, further comprising a first balloon on a distal portion of the catheter.

6. The system of claim 5, wherein the catheter includes one or more openings fluidly coupled to a source of acoustic coupling medium, wherein acoustic coupling medium is injected into the duodenum and retained in the duodenum by the first balloon.

7. The system of claim 6, further comprising a second balloon on a proximal portion of the catheter and configured to retain the acoustic coupling medium between the first balloon and second balloon.

8. A histotripsy catheter system, comprising:
a therapy transducer configured to emit an ultrasound signal and generate a bubble cloud at a focal point of the therapy transducer;
a balloon configured to surround the therapy transducer, the balloon in fluid communication with a source of acoustic coupling medium, wherein receipt of acoustic coupling medium into the balloon inflates the balloon against a mucosa of a duodenum of a patient and acoustically couple the therapy transducer with the mucosa of the duodenum.

9. The histotripsy catheter system of claim 8, wherein the therapy transducer is an omnidirectional therapy transducer configured for electronic focusing and steering of the focal point to the mucosa of the duodenum of the patient.

10. The histotripsy catheter system of claim 8, wherein the therapy transducer is a single directional therapy transducer configured for mechanical rotation and electronic focusing and steering of the of the focal point to the mucosa of the duodenum of the patient.

11. The histotripsy catheter system of claim 8, further comprising an application stored in a memory and when executed by a processor of a computing device cause the computing device to:
receive images of a duodenum of a patient;
define an inner layer of the duodenum;
define a therapy cylinder;
define a plurality of bubble cloud locations matching a periphery of the therapy cylinder; and
apply histotripsy therapy from the therapy transducer to form a bubble cloud at each of the defined bubble cloud locations.

12. A histotripsy catheter system, comprising:
a therapy transducer configured to emit an ultrasound signal and generate a bubble cloud at a focal point of the therapy transducer;
a biasing means configured to bias the therapy transducer against a mucosa of a duodenum and acoustically couple the therapy transducer with the mucosa of the duodenum.

13. The histotripsy catheter system of claim 12, further comprising a vacuum source in fluid communication with the therapy transducer, wherein application of vacuum to the therapy transducer suctions the therapy transducer to the mucosa of the duodenum.

14. A method of apply histotripsy to a duodenum of a patient comprising:
acquiring images of the duodenum;
confirming acoustic coupling of the duodenum with a therapy transducer;
define an inner layer of tissue of the duodenum
define a therapy cylinder;
define a plurality of bubble cloud locations matching a periphery of the therapy cylinder; and
applying histotripsy therapy to form a bubble cloud at each of the defined bubble cloud locations.

15. The method of claim 14, further comprising robotically driving the therapy transducer to a position and orientation where a focal point of the therapy transducer matches the bubble cloud locations.

16. The method of claim 14, wherein the acquired images are ultrasound images, fluoroscopic images, or cone-beam computed tomography images.

17. The method of claim 14, wherein the therapy cylinder, bubble cloud locations, and positions and orientations of a therapy transducer define a therapy plan.

18. The method of claim 17, further comprising:
adjusting the therapy plan.

19. The method of claim 14, further comprising inserting a catheter into the duodenum of the patient.

20. The method of claim 19, further comprising inflating at least one balloon on the catheter.
